(19) United States
(12) Patent Application Publication
(10) Pub. No.: US 2008/0294145 A1
(43) Pub. Date: Nov. 27, 2008

(54) CATHETER HUB WITH FLUSHABLE LUMEN AND GUIDEWIRE

(75) Inventors: James R. Eddings, Garland, TX (US); David George Catlin, Heath, TX (US); Michael Glen Vaughn, Garland, TX (US)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: GALT MEDICAL CORPORATION, Garland, TX (US)

(21) Appl. No.: 11/753,781

(22) Filed: May 25, 2007

(51) Int. Cl. A61M 25/18

(52) U.S. Cl. 604/533

(57) ABSTRACT

A connector for a catheter assembly, the connector comprising a hub having a body comprising a first section and a second section, a first opening in the first section sized to receive a male connector, a collar associated with the second section, a passage extending through the first section and having a second opening suitable for conducting fluid therethrough; a tube section disposed within the second section, the tube section having a first lumen for accommodating a guidewire and a second lumen for conducting a flushing fluid therethrough, the second lumen being in fluid communication with the passage; and, a guidewire.

### FIELD OF THE INVENTION

The present invention relates generally to flushable catheter assemblies which incorporate a guidewire.

### BACKGROUND OF THE INVENTION

Catheters, such as peripherally inserted central catheters (“PICC”), have long been used for providing long term access to the vascular system of a patient. The typical principal components of a PICC assembly (sometimes referred to as a “PICC line”) include a (female) catheter hub for leakless escapement of a catheter to a component of the catheter assembly, a syringe, and a guidewire attached to a guidewire handle. A guidewire is commonly used in a PICC assembly to stiffen the catheter and facilitate handling and manipulation of the catheter during insertion. To help minimize the risk of vessel perforation or trauma with the use of guidewires the proximal end of the guidewire typically has a soft flexible tip or a ball end. Proper placement of the guidewire requires that the proximal tip is a short length inside the proximal tip of the catheter to protect against vessel wall puncture.

The term “distal” as used herein shall mean the portion closest to the tip of the guidewire to be inserted into a patient's body and the term “proximal” shall mean the portion closer to the hub assembly access opening. During catheter placement, it is occasionally necessary to adjust the length of the catheter by cutting the catheter. During the catheter length adjustment step, the guidewire threaded inside the catheter must be slightly retracted or removed from the catheter in order avoid cutting off a portion of the soft proximal tip of the guidewire.

It is typically recommended to flush the catheter with an aqueous solution, e.g., saline or heparin, before, during and after insertion. Flushing the catheter prior to insertion checks for catheter patency. Flushing during installation to facilitate maneuvering and prevent blood from entering the catheter assembly and clotting. Flushing after installation is done to facilitate removal of the guidewire. Accordingly, there is a need for a PICC assembly which allows for flushing in a convenient, efficient manner and one which reduces the amount of user handling or manipulation.

During catheter insertion and removal it is desirable to flush the inside of the catheter with a flushing solution to help reduce the friction between the inside catheter wall and the guidewire. It is also desirable to remove any air trapped in the catheter line. It would also be desirable to have a catheter/guidewire assembly that is more efficiently able to be inserted through valves in the vasculature while minimizing trauma to the valves.

It would be desirable to have the flushing port substantially parallel to the guidewire insertion port to make the assembly less bulky and affording it a substantially in-line construction. A more compact in-line design is less likely to snag, dislodge the catheter hub and cause discomfort to the patient. Further, it would be desirable to have a one-piece connector for a flushable guidewire assembly.

### SUMMARY OF THE INVENTION

One exemplary embodiment of the present invention provides a connector for a catheter assembly, comprising a hub having a body comprising a first section and a second section, a first opening in the first section sized to receive a male connector, a collar associated with the second section, a passage extending through the first section and having a second opening suitable for conducting fluid therethrough; a tube section disposed within the second section, the tube section having a first lumen for accommodating a guidewire and a second lumen for conducting a flushing fluid therethrough, the second lumen being in fluid communication with the passage; and, a guidewire.

Other features of the present invention will become apparent upon reading the following detailed description of exemplary embodiments of the invention, when taken in conjunction with the appended claims.

### BRIEF DESCRIPTION OF THE DRAWINGS

Exemplary embodiments of the present invention are illustrated in the drawings in which like reference characters designate the same or similar parts throughout the figures of which:

FIG. 1 is a cross-sectional side perspective view of a first exemplary embodiment of hub assembly with guidewire.

FIG. 2 is a perspective view of a dual-lumen tube used in the first exemplary embodiment of FIG. 1.

FIG. 3 is a cross-sectional end view of the dual lumen tube of FIG. 2.

FIG. 4 is a side view of the dual lumen tube of FIG. 2.

FIG. 5 is a cross-sectional side view of the dual lumen tube of FIG. 2.

FIG. 6A is a cross-sectional side perspective view of a first alternative exemplary embodiment of hub assembly with guidewire.

FIG. 6B is a perspective view of the dual lumen tube of FIG. 6A.

FIG. 6C is an end view of the dual lumen tube of FIG. 6A.

FIG. 6D is a side view of the dual lumen tube of FIG. 6A.

FIG. 6E is a side cutaway view of the dual lumen tube of FIG. 6A.

FIG. 7 is a cross-sectional side perspective view of a second alternative exemplary embodiment of hub assembly with guidewire.

FIG. 8 is a cross-sectional side perspective view of the second alternative embodiment with the adjustable guidewire in the advanced position.

FIG. 9 is a cross-sectional side perspective view of the second alternative embodiment with the adjustable guidewire in the retracted position.

FIG. 10 is a cross-sectional end view of an alternative embodiment of a dual-lumen tube.

FIG. 11 is a cross-sectional side view of an alternative embodiment of a dual lumen tube.

DETAILED DESCRIPTION OF THE PREFERRED EMBODIMENTS

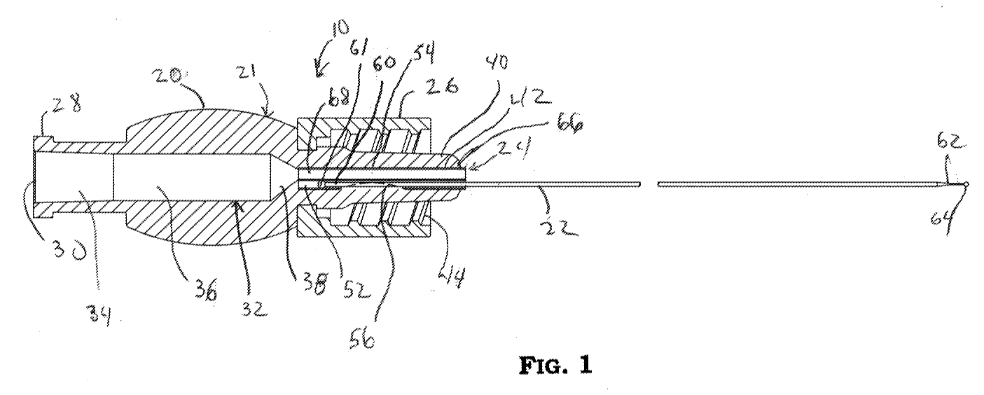

FIG. 1 shows a first exemplary embodiment of an assembly 10 comprising generally a hub 20, a guidewire 22, a dual-lumen tube 24 and a collar 26.

The hub 20 may be made of any suitable bioinert moldable material, such as, but not limited to, high density polyethylene (“HDPE”), metal, plastic, glass, ceramic, alloy, composite, combinations and mixtures thereof or other material. The hub 20 preferably has an outer flange 28. The hub 20 has a first section 21 having an opening 30 and a passage 32. The passage 32 has a preferably slightly tapered first passage section 34, and the opening 30 and passage 32 may form a female Luer lock or Luer slip which may accommodate a male Luer lock or Luer slip. Alternatively, the flange 28, opening 30 and/or passage 32 may accommodate other connection mechanism known to those skilled in the art or developed hereafter (not shown). The passage 32 has a second section 36 and a tapered third portion 38. The hub 20 has a second section 40 having a bore 42 extending therethrough. The collar 26 is associated with the exterior of the hub second section 40. The collar 26 preferably has a set of internal threads 44 which can mate with a conventional PICC line. The collar 26 may be a conventional male Luer lock or Luer slip or other connection mechanism known to those skilled in the art or developed hereafter.

Instead of a syringe, fluid may be delivered to the passage 32 by means of a tube, fluid reservoir and a mechanism for pressurizing the fluid. The fluid reservoir may be a bulb, bag, syringe, or other reservoir. The pressurizing mechanism may be a syringe plunger, bulb, motor or the like. The pressurizing mechanism may have an actuator, such as a switch or button for activating and deactivating the pump. The actuator may have a mechanical or electromechanical control for controlling the pressure and amount of fluid delivered to the hub 20.

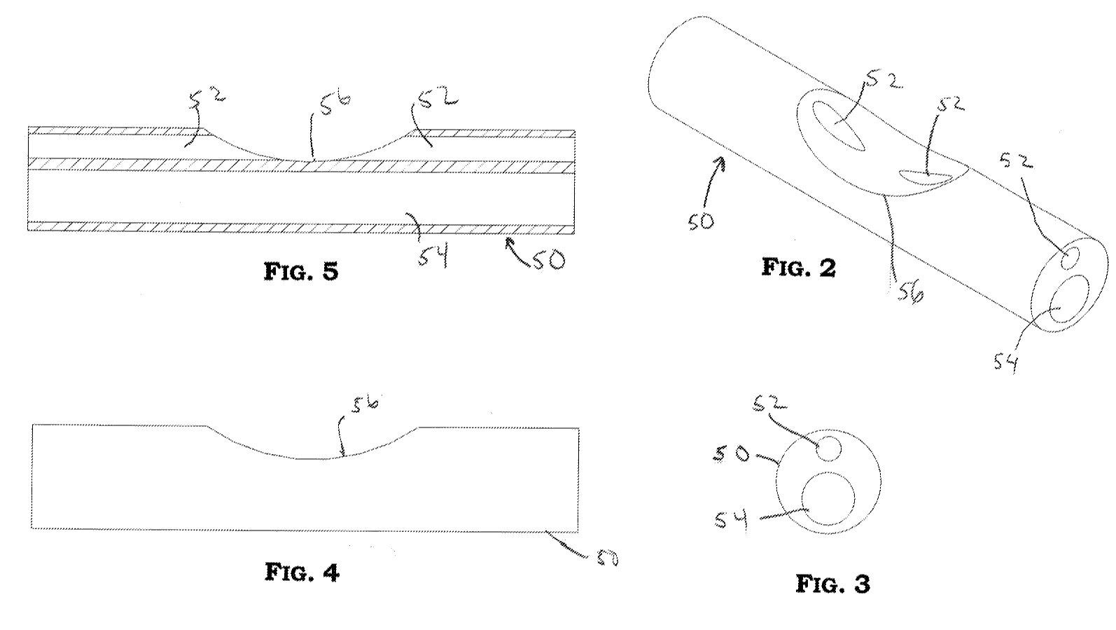

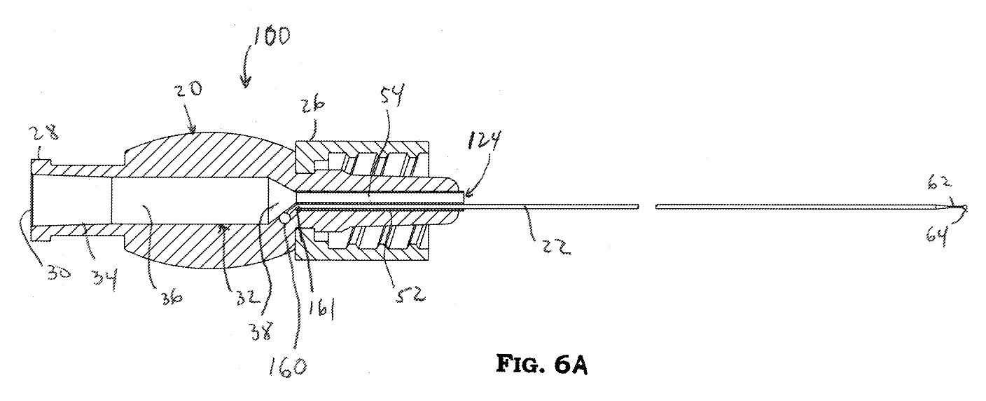

As shown in one exemplary embodiment in FIGS. 2-5, the bore 42 accommodates a dual-lumen tube 24, which is associated with the bore 42 during the molding process. The tube 24 comprises a guidewire-receiving lumen 52 (also referred to as the guidewire lumen 52) and a flush lumen 54. The guidewire lumen 52 is preferably in a generally parallel position with respect to the flush lumen 54. Alternatively, it is possible for the two lumens 52, 54 to be at slight angles with respect to each other. For the purposes of the present disclosure, the term “co-parallel” is intended to mean parallel to or at a slight angle. In one embodiment the flush lumen 54 has a larger diameter than the guidewire lumen 52. In another embodiment, the flush lumen 54 may be of the same or smaller diameter compared to the guidewire lumen 52. Preferably, the tube 24 has a portion of the body skived or shaved away (shown at skived portion 56). The skived portion 56 may be achieved by, for example, molding the tube 24 with the skived portion 56 formed therein, or, forming a whole tube 24 and shaving away or otherwise removing a portion of the tube 24. The purpose of the skived portion 56 is to expose a portion of the guidewire lumen 52 (the purpose of the exposed portion will be discussed in greater detail hereinbelow).

The guidewire 22 preferably has a proximal end 60 and has a distal end terminating in a tip 62. The tip 62 is preferably flexible or soft and may have a rounded end 64 so that it may be inserted into a blood vessel and advanced within the vessel without puncturing the vessel. The guidewire 22 can be made of a stiffened but flexible material, such as, but not limited to, metal or plastic wire, and may be formed as a solid core, coil, braid, multiple layer, coaxial, combinations of the foregoing or other construction. The guidewire 22 may be hydrophilic or coated with silicone or other material to reduce friction. The proximal end 60 preferably, though not mandatorily, has an anchor portion 61 which is preferably expanded in diameter or can otherwise provide a surface about which plastic material can form and prevent or reduce the likelihood of the guidewire 22 slipping within the guidewire lumen 52. The anchor portion 61 is preferably ball-shaped, but alternatively may be a flattened expanded diameter, T-shaped, reverse tapered, hook, loop, blade, ellipsoid, toroid, combinations thereof or have another shape. The anchor portion 61 is preferably at the tip of the proximal end 60, but, alternatively, may be in proximity to the tip, i.e., slightly in from the proximal end 60 toward the distal end 62.

The hub assembly 10 may be constructed in any of several way. One construction method is to form the tube 24, then insert the guidewire 22, tip 62 end first into the guidewire lumen 52 so that the end 60 is within the tube 24 and the tip 62 end extends beyond the tube 24. The tube 24 can be then molded into the hub second section 40 during the process in which the hub 20 is molded. During the molding process plastic material flows into the skived portion 56 and the guidewire lumen 52 and contacts both the tube 24 and the guidewire 22 so as to seal the guidewire 22 within the guidewire lumen 52 and prevent movement of the guidewire 22 within the guidewire lumen 52. The tube 24 preferably extends slightly beyond the distal end 66 of the hub second section 40.

An alternative method of manufacturing the assembly 10 is to first insert the guidewire 22 into the tube 24 as described hereinabove. The guidewire 22 can then be locked into place within the tube 24 by forming, ultrasonic welding, gluing or the like. The hub 20 can be molded as two separate halves and the tube 24 (with the guidewire 22 locked therein) can then be snap-fitted into one half the hub second section 40. The two halves can then be joined and sealed, such as by gluing, sonic welding, fusing, or other technique known to those skilled in the art for joining two parts of a molded plastic device.

For both methods of manufacture described above, when assembly is complete, the flush lumen 54 has a proximal end 68 in fluid communication with the third section 38 of the hub 20.

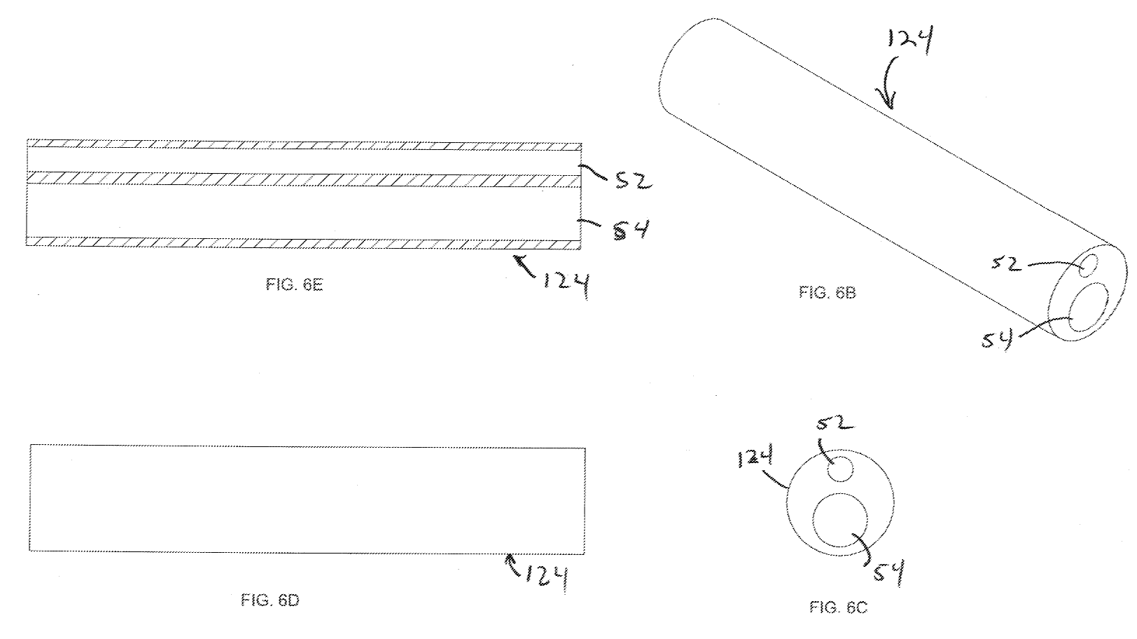

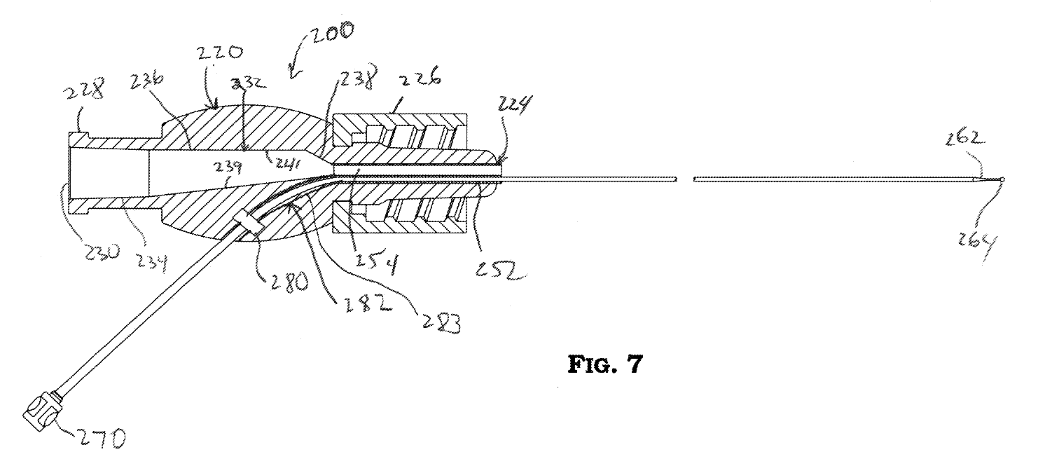

FIGS. 6A-6E shows a first alternative exemplary embodiment of a hub assembly 100, similar in most respects to the hub assembly 10, with the following distinctions. In this embodiment, the tube 124, as shown in FIGS. 6B, 6D and 6E, is not skived. During manufacture, the guidewire tip 62 is inserted into the tube 24 guidewire lumen 52, as described hereinabove; however, the ball end 160 of the guidewire 22 remains outside of the tube 24 and a portion 161 of the guidewire 22 is bent to prevent slippage of the guidewire 22 within the guidewire lumen 52. The tube 24 and guidewire are molded with the hub 20 and the ball end 160 is maintained in position within the hub 20.

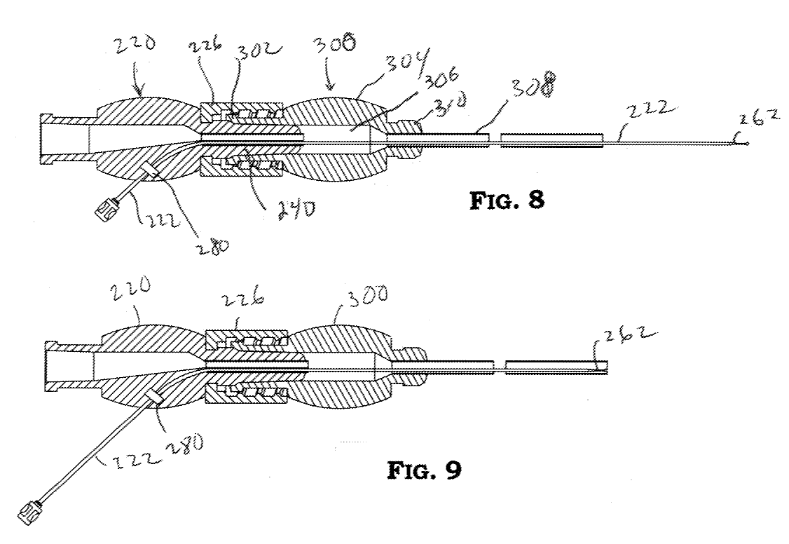

FIGS. 7-9 show a second alternative exemplary embodiment of a hub assembly 200 with an adjustable guidewire and having a hub 220, a hub first section 221, guidewire 222, dual lumen tube 224, collar 226, flange 228 and an opening 230. A passage 232 has a first section 234, second section 236 and third section 238. The second section 234 preferably tapers, and, preferably with the lower wall portion 239 tapering toward the upper wall portion 241. The third section 238 has the upper wall portion 241 tapering more sharply toward the lower wall portion 239.

The tube 224 has a guidewire lumen 252 and a flush lumen 254. The guidewire 222 has a tip 262 preferably with a rounded end 264. At the proximal portion of the guidewire 222 (opposite the tip 262), the guidewire has a handle 270. The handle may comprise a knob, T-bar, knurled nut, loop, ball, ellipsoid, toroid or other regular or irregular shape sufficient to enable a user to grasp the guidewire and advance or retract it within the hub 220.

The hub assembly 200 is manufactured having the valve seal 280 inserted into the mold of the hub 220. The valve seal 280 may be a conventional silicone rubber or other septum or fluid tight seal having an aperture to accommodate the guidewire 222, as is known to those skilled in the art. In one exemplary embodiment the guidewire 222 is first inserted through the valve seal 280 and both the guidewire 222 and valve seal 280 are then positioned within the mold cavity and held in position. The plastic is injected into the mold and the guidewire 222 portion within the mold is encapsulated by the plastic. After solidification the guidewire 222 may still slide within the hub assembly 20 because the guidewire 222 is smooth (and, may optionally be coated with a lubricant) and the injection plastic may have waxy release agents which allow for the guidewire 222 to slide within the plastic.

In an alternative method of manufacturing the hub assembly 200, a channel 282 is formed as part of the mold. The channel 282 preferably has a curved portion 283 to prevent kinking of the guidewire 222 during insertion and manipulation. The valve seal 280 is inserted into the mold cavity inline with the channel 282 and held in position while plastic is injected into the mold. After the hub 220 is molded, the guidewire tip 262 is inserted through the valve seal 280 and the channel 282 and through the guidewire lumen 252. The guidewire 222 can slide within the channel 282, valve seal aperture (not shown), and guidewire lumen 252. Preferably, the guidewire 222 is coated with a friction resistance material, such as silicone or the like, as is commonly used in the art. The tube 224 is preferably a distinct component which is molded into the hub 220 during manufacture. The third section 238 is in fluid communication with the flush lumen 254.

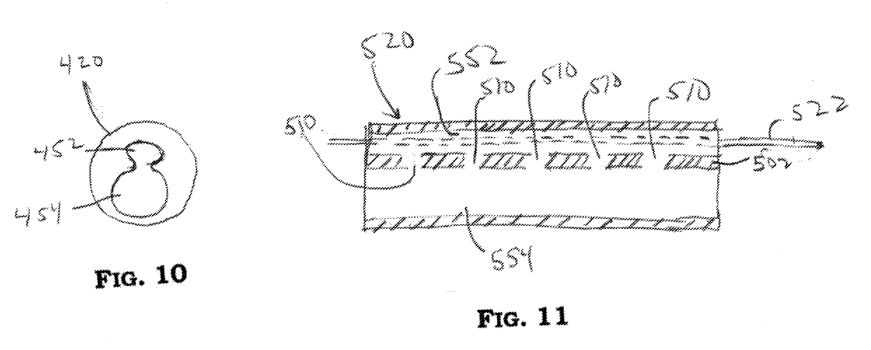

In this embodiment the guidewire 222 can be manipulated and slid within the hub 220 by pulling or pushing on the handle 270 to advance or retract the guidewire 222. FIG. 8 shows the guidewire 222 in an advanced position and FIG. 9 shows the guidewire 222 in an retracted position.

In all three of the above-described exemplary embodiments the opening 30 and 230 can accommodate a syringe tip or other connector associated with a device (e.g., syringe barrel, squirt bulb, motorized fluid delivery pump system, or the like) for introducing fluid into and through the passage 32 or 232 and into and through the flush lumen 54 or 254. The fluid can then be used to flush a PICC line 300 attached to the collar 26 or 226 (as shown in FIGS. 8-9). The PICC line 300 is a conventional catheter having a male collar 302 that mates and locks with the collar 226. A catheter hub 304 has a passage 306 which sized to receive the hub second section 40 or 240. A catheter 308 extends from the distal end 310 of the hub 304. The catheter 308 receives the guidewire 22 or 222.

In operation of the second alternative exemplary embodiment the catheter 308 is measured to the approximate length desired for the particular patient and then the excess catheter is cut. The guidewire 222 is then retracted (as shown in FIG. 9) so that the tip 262 is generally proximate to the end of the catheter 308. Thus, when the catheter 308 and guidewire 222 are inserted into the patient, such as, but not limited to, during a Seldinger procedure, the guidewire 222 provides the requisite stiffness to the catheter 308 to ensure proper insertion and positioning.

Commonly, the PICC line 300 is flushed prior to insertion into the patient's blood vessel, possibly during insertion, and as needed after insertion. The flushable lumen 54 or 254 enables the flushing of the catheter 308 without the need for disconnecting the hub 20 or 220 from the PICC line 300. Thus, the user saves time and effort, reduces the risk of infection and reduces the risk of vessel puncture by reducing the manipulation of the assembly 20 or 220.

The first exemplary embodiment (FIGS. 1-5) and the first alternative exemplary embodiment (FIG. 6) are flushable in the same manner as the second alternative embodiment (FIGS. 7-9) but do not provide the adjustability of the guidewire 22 length.

In a fourth exemplary embodiment a hub assembly may be constructed similar to either the assembly 100 or 200, but instead of the dual-lumen tubes 24 or 224, a dual-lumen tube 420, shown in cross-section view in FIG. 10 may be employed. The tube 420 has distinct lumens 452, 454, but has an open space between the lumens, allowing for some flushing fluid to potentially be in contact with the guidewire (not shown, but is as described hereinabove).

In a fifth exemplary embodiment, shown in FIG. 11, the hub assembly is similar to the fourth exemplary embodiment, however, a dual-lumen tube 520 has a guidewire lumen 552 (which accommodates a guidewire 522) and a flush lumen 554. A wall 502 between the lumens 552, 554 has openings 510, which may be perforations, slots, grid, mesh, or other type of aperture. The openings 510 allow for at least partial fluid communication between the lumens.

The present invention provides a number of advantages over non-flushable catheter assemblies, several nonlimiting examples of which are discussed hereinbelow. The present invention allows for quick, efficient flushing of the catheter prior to, during and after insertion. Flushing prior to insertion helps reduce friction between the guidewire 222 and the inside wall of the catheter 308. Pre-flushing also helps remove any air bubbles which may be trapped in the catheter 308. Flushing during insertion can help open any impeding valves in the vasculature and reduce trauma to the valve during insertion of the catheter. Flushing after insertion also helps remove any residual air bubbles.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. It should further be noted that any patents, applications and publications referred to herein are incorporated by reference in their entirety.

Claimed is:

1. A connector for a catheter assembly, comprising:
a. a hub having
i. a body comprising a first section and a second section,
ii. a first opening in said first section sized to receive a male connector,
iii. a collar associated with said second section,
iv. a passage extending through said first section and having a second opening suitable for conducting fluid therethrough;
b. a tube section disposed within said second section, said tube section having a first lumen for accommodating a guidewire and a second lumen for conducting a flushing fluid therethrough, said second lumen being in fluid communication with said passage; and,
c. a guidewire.

2. The connector of claim 1, wherein said first and second lumens being in a substantially co-parallel relationship.

3. The connector of claim 1, wherein said guidewire has a proximal end terminating in an expanded diameter portion and a distal end terminating in a tip.

4. The connector of claim 1, wherein said expanded diameter portion has a shape selected from the group comprising a ball, ellipsoid, toroid, “T”, loop, hook, and blade.

5. The connector of claim 1, wherein said collar further comprises a connector.

6. The connector of claim 5, further comprising a catheter comprising an elongated tube and a catheter connector suitable for being associated with said collar connector.

7. The connector of claim 1, wherein at least a portion of said passageway tapers toward said second opening.

8. The connector of claim 1, wherein said guidewire has a proximal end terminating in an expanded diameter portion and a distal end terminating in a tip, said expanded diameter portion being disposed within said first lumen.

9. The connector of claim 8, wherein a portion of tube section proximate to said first lumen includes a recessed area having a portion of said first lumen exposed.

10. The connector of claim 1, wherein said guidewire has a proximal end terminating in an expanded diameter portion and a distal end terminating in a tip, said expanded diameter portion being disposed substantially outside of said first lumen and within said hub.

11. The connector of claim 1, further comprising a channel formed within said hub, said channel sized to accommodate said guidewire.

12. The connector of claim 11, wherein said channel has a first end having a proximal opening out of said hub first section.

13. The connector of claim 11, wherein said channel has a second end having a distal opening in communication with said first opening in said tube.

14. The connector of claim 11, wherein at least a portion of said channel is curved.

15. The connector of claim 11, further comprising a valve seal disposed within said first section and collinear with said channel.

16. The connector of claim 11, wherein said guidewire further comprises a proximal end having a handle associated therewith.

17. The connector of claim 11, wherein at least a portion of said guidewire is slidingly disposed within said channel and within said first lumen.

18. The connector of claim 15, wherein at least a portion of said guidewire is slidingly disposed within said channel, said valve seal and within said first lumen.

19. The connector of claim 6, wherein said guidewire is at least partially disposed within said catheter.

20. The connector of claim 11, wherein said catheter can conduct a flushing fluid introduced at said hub first section opening and through said passage and said second lumen into said catheter.

21. The connector of claim 1, wherein said first opening comprises a Luer lock.

22. The connector of claim 1, wherein said first opening comprises a Luer slip.

23. The connector of claim 1, wherein said collar comprises a Luer lock.

24. The connector of claim 1, wherein said collar comprises a Luer slip.

25. A flushable catheter assembly, comprising:
a. a hub having
i. a body comprising a first section and a second section,
ii. a first opening in said first section sized to receive a male connector,
iii. a collar associated with said second section,
iv. a passage extending through said first section and having a second opening suitable for conducting fluid therethrough;
b. a tube section disposed within said second section, said tube section having a first lumen for accommodating a guidewire and a second lumen for conducting a flushing fluid therethrough, said second lumen being in fluid communication with said passage;
c. a guidewire; and,
d. a catheter.

26. The flushable catheter assembly of claim 25, wherein said catheter comprises an elongated tube and a connector matable with said collar.

27. The flushable catheter assembly of claim 25, further comprising a means for delivering a pressurized flow of flushing liquid to said hub.

28. The flushable catheter assembly of claim 27, wherein said means for delivering a pressurized flow of flushing liquid comprises a connector connectable to said first opening in said hub first section, a fluid reservoir and a means for pressurizing said fluid.

29. The flushable catheter assembly of claim 27, wherein said means for delivering a pressurized flow of flushing liquid comprises a syringe connectable to said first opening in said hub first section.

30. The flushable catheter assembly of claim 27, wherein said means for delivering a pressurized flow of flushing liquid comprises a fluid reservoir and a pump associated with said fluid reservoir.

31. The flushable catheter assembly of claim 30, further comprising an actuator for actuating said pump.

32. The flushable catheter assembly of claim 30, further comprising a control for controlling the pressure of the fluid and the amount of fluid delivered to said hub.

33. The connector of claim 1, wherein said tube section first and second lumens are at least partially in fluid communication with each other.

34. The connector of claim 1, wherein said tube section first and second lumens share a common wall, said wall having at least one opening allowing for at least partial fluid communication between said first and second lumens.

35. A catheter assembly for flushing a catheter with a guidewire, comprising:
a. a hub having
i. a body comprising a first section and a second section,
ii. a first opening in said first section sized to receive a male connector,
iii. a collar associated with said second section,
iv. a passage extending through said first section and having a second opening suitable for conducting fluid therethrough;
b. a tube section disposed within said second section, said tube section having a first lumen for accommodating a guidewire and a second lumen for conducting a flushing fluid therethrough, said second lumen being in fluid communication with said passage;
c. a guidewire;
d. a catheter; and,
e. a syringe suitable for containing a flushing fluid and connectable to said first opening in said hub first section.

36. A method of flushing a catheter utilizing an inline flushable catheter hub, comprising:
a. providing a catheter assembly for flushing a catheter with a guidewire, comprising:
i. a hub having
1. a body comprising a first section and a second section,
2. a first opening in said first section sized to receive a male connector,
3. a collar associated with said second section,
4. a passage extending through said first section and having a second opening suitable for conducting fluid therethrough;
ii. a tube section disposed within said second section, said tube section having a first lumen for accommodating a guidewire and a second lumen for conducting a flushing fluid therethrough, said second lumen being in fluid communication with said passage;
iii. a guidewire;
iv. a catheter; and,
v. a syringe suitable for containing a flushing fluid and connectable to said first opening in said hub first section;
b. controllably introducing a flushing fluid into said passage, through said second lumen and into said catheter.

37. A connector for a flushable catheter assembly, comprising:
a. a distal end opening for removable, leakless attachment to a syringe;
b. a substantially co-parallel proximal end opening for removable, leakless attachment to a catheter hub;
c. a guidewire opening non-co-parallel with said distal end opening and said proximal end opening;
d. a flush lumen for flushing the flushable catheter assembly;
e. a guidewire lumen, the flush lumen and the guidewire lumen substantially co-parallel with said distal end opening and said proximal end opening; and,
f. a guidewire slidably pre-disposed inside the guidewire lumen and said guidewire opening, the guidewire lumen in communication with the guidewire opening, the guidewire adjustable via movement of its distal end protruding from the guidewire opening.

38. A method for manufacturing a one-piece connector for a flushable catheter assembly with guidewire comprising the step of injection molding of a dual-lumen tube inside of a hub, the dual-lumen tube comprising a flush lumen and a guidewire lumen.

39. A method for cutting a catheter which is part of a flushable catheter assembly to desired length, comprising the steps of:
a. providing a connector for a flushable catheter assembly, said connector comprising a distal end opening for removable, leakless attachment to a syringe, a substantially co-parallel proximal end opening for attachment to a catheter hub, a guidewire opening non-co-parallel with said distal end opening and said proximal end opening, a flush lumen for flushing said flushable catheter assembly, a guidewire lumen, said flush lumen and said guidewire lumen substantially co-parallel with said distal end opening and said proximal end opening, a guidewire slidably pre-disposed inside the guidewire lumen and said guidewire opening, said guidewire lumen in communication with said guidewire opening, said guidewire adjustable via movement of said distal end protruding from said guidewire opening;
b. threading the guidewire inside said catheter by attaching said proximal opening of said connector to said catheter hub;
c. attaching the distal opening of said connector to a syringe containing a flush liquid;
d. depressing the plunger of said syringe to flush said flushable catheter assembly;
e. manually advancing or withdrawing said guidewire to a desired position inside said catheter; and,
f. cutting said catheter to a desired length.
